(19) United States
(12) Patent Application Publication
(10) Pub. No.: US 2004/0186155 A1
(43) Pub. Date: Sep. 23, 2004

(54) Combination therapy for the treatment or prevention of migraine

(76) Inventors: Jeffrey Marc Dayno, Maple Glen, PA (US); Wim Vandenhouweele, Whitehouse Station, NJ (US)

Correspondence Address: MERCK AND CO INC, P O BOX 2000, RAHWAY, NJ, 070650907

(21) Appl. No.: 10/767,025

(22) Filed: Jan. 29, 2004

Related U.S. Application Data
(60) Provisional application No. 60/443,701, filed on Jan. 30, 2003.

(51) Int. Cl. A61K 31/415; A61K 31/365

(52) U.S. Cl. 514/406; 514/471

(57) ABSTRACT

The present invention encompasses a method for treating or preventing migraine in a mammalian patient in need thereof comprising concomitantly or sequentially administering to said patient a cyclooxygenase-2 selective inhibitor and a beta adrenergic receptor blocking agent in amounts that are effective for treating or preventing migraine. Pharmaceutical compositions are also included.

### BACKGROUND OF THE INVENTION

Migraines are recurrent, often familial, symptom complexes of periodic attacks of vascular headache. The condition is characterized by intermittent attacks of headache, preceded by an aura in approximately 15% of patients. The headache is often accompanied by associated symptoms, most commonly nausea, vomiting, photophobia and phonophobia. Migraines affect approximately 17% of adult women and 6% of adult men (Stewart et al., Neurology, 1994, 44 (suppl. 4), 517-523).

Selective inhibitors of cyclooxygenase-2 are a sub-class of the class of drugs known as non-steroidal antiinflammatory drugs (NSAIDs). The NSAIDs are active in reducing the prostaglandin-induced pain and swelling associated with the inflammation process but are also active in affecting other prostaglandin-regulated processes not associated with the inflammation process. Thus, use of high doses of most common NSAIDs can produce severe side effects, including life threatening ulcers, that limit their therapeutic potential. An alternative to NSAIDs is the use of corticosteroids, which have even more drastic side effects, especially when long term therapy is involved.

Previous NSAIDs have been found to prevent the production of prostaglandin by inhibiting enzymes in the human arachidonic acid/prostaglandin pathway including the enzyme cyclooxygenase (COX). The discovery that there are two isoforms of the COX enzyme, the first, COX-1, being involved with physiological functions and the second, COX-2, being induced in inflamed tissue, has given rise to a new approach. While conventional NSAIDs block both forms of the enzyme, the identification of the inducible COX-2 enzyme associated with inflammation has provided a viable target of inhibition which more effectively reduces inflammation and produces fewer and less drastic side effects. Many compounds which have activity as COX-2 inhibitors have been identified, including rofecoxib (VIOXX®), etoricoxib (ARCOXIA™), celecoxib (CELEBREX®) and valdecoxib (BEXTRA™), and much research continues in this area.

A variety of beta adrenergic blocking agents are known in the art, including acebutolol, atenolol, betaxolol, bioprolol, carteolol, labetalol, metoprolol, nadolol, penbutolol, pindolol, propanolol, and timolol, and are employed in migraine preventive therapy. Timolol maleate (BLOCADREN®) is indicated for the propylaxis of migraine headache.

The present invention is directed to a novel therapy for the treatment and prevention of migraine headache utilizing a combination therapy of a cyclooxygenase-2 selective inhibitor, such as rofecoxib, and a beta adrenergic receptor blocking agent, such as timolol.

### SUMMARY OF THE INVENTION

The present invention encompasses a method for treating or preventing migraine in a mammalian patient in need thereof comprising concomitantly or sequentially administering to said patient a cyclooxygenase-2 selective inhibitor and a beta adrenergic receptor blocking agent in amounts that are effective for treating or preventing migraine. Pharmaceutical compositions are also included.

### DETAILED DESCRIPTION OF THE INVENTION

The present invention encompasses a method for treating or preventing migraine in a mammalian patient in need thereof comprising concomitantly or sequentially administering to said patient a cyclooxygenase-2 selective inhibitor and a beta adrenergic receptor blocking agent in amounts that are effective for treating or preventing migraine.

In an embodiment of the invention the cyclooxygenase-2 selective inhibitor is selected from the group consisting of: rofecoxib, etoricoxib, celecoxib, valdecoxib, parecoxib, lumiracoxib, BMS347070, tiracoxib, ABT963, CS502 and GW406381.

Within this embodiment of the invention the cyclooxygenase-2 selective inhibitor is rofecoxib. Also within this embodiment of the invention the cyclooxygenase-2 selective inhibitor is rofecoxib administered at a dose of about 12.5 mg or about 25 mg.

Within this embodiment of the invention the cyclooxygenase-2 selective inhibitor the cyclooxygenase-2 selective inhibitor is etoricoxib. Also within this embodiment of the invention the cyclooxygenase-2 selective inhibitor is etoricoxib administered at a dose of about 60 mg, about 90 mg or about 120 mg.

Within this embodiment of the invention the cyclooxygenase-2 selective inhibitor the cyclooxygenase-2 selective inhibitor is celecoxib. Also within this embodiment of the invention the cyclooxygenase-2 selective inhibitor is celecoxib administered at a dose of about 100 mg or about 200 mg or about 400 mg.

Within this embodiment of the invention the cyclooxygenase-2 selective inhibitor the cyclooxygenase-2 selective inhibitor is valdecoxib. Also within this embodiment of the invention the cyclooxygenase-2 selective inhibitor is valdecoxib administered at a dose of about 10 mg or about 20 mg.

In another embodiment of the invention the beta adrenergic receptor blocking agent is selected from the group consisting of: acebutolol, atenolol, betaxolol, bioprolol, carteolol, labetalol, metoprolol, nadolol, penbutolol, pindolol, propanolol, and timolol, or pharmaceutically acceptable salts thereof. Within this embodiment the beta adrenergic receptor blocking agent is timolol maleate. Also within this embodiment of the invention the beta adrenergic receptor blocking agent is timolol maleate administered at a dose of about 5 mg, about 10 mg or about 20 mg.

Another embodiment of the invention encompasses a method for treating migraine in a mammalian patient in need thereof comprising concomitantly or sequentially administering to said patient a cyclooxygenase-2 selective inhibitor and a beta adrenergic receptor blocking agent in amounts that are effective for treating migraine. Within this embodiment the cyclooxygenase-2 selective inhibitor is rofecoxib and the beta adrenergic receptor blocking agent is timolol maleate.

Another embodiment of the invention encompasses a method for preventing migraine in a mammalian patient in need thereof comprising concomitantly or sequentially administering to said patient a cyclooxygenase-2 selective inhibitor and a beta adrenergic receptor blocking agent in amounts that are effective for preventing migraine. Within this embodiment the cyclooxygenase-2 selective inhibitor is rofecoxib and the beta adrenergic receptor blocking agent is timolol maleate.

The invention also encompasses a pharmaceutical composition comprising a cyclooxygenase-2 selective inhibitor and a beta adrenergic receptor blocking agent in combination with a pharmaceutically acceptable carrier. Within this embodiment the cyclooxygenase-2 selective inhibitor is rofecoxib and the beta adrenergic receptor blocking agent is timolol maleate.

The terms “inhibitor of cyclooxygenase-2”, “cyclooxygenase-2 selective inhibitor” and “COX-2 inhibitor” as used herein embrace compounds which selectively inhibit cyclooxygenase-2 over cyclooxygenase-1, including pharmaceutically acceptable salts thereof. Employing the human whole blood COX-1 assay and the human whole blood COX-2 assay described in C. Brideau et al, Inflamm. Res. 45: 68-74 (1996), herein incorporated by reference, preferably, the compounds have a cyclooxygenase-2 IC₅₀ of less than about 2 μM in the human whole blood COX-2 assay, yet have a cyclooxygenase-1 IC₅₀ of greater than about 5 μM in the human whole blood COX-1 assay. Also preferably, the compounds have a selectivity ratio of cyclooxygenase-2 inhibition over cyclooxygenase-1 inhibition of at least 10, and more preferably of at least 40. The resulting selectivity may indicate an ability to reduce the incidence of common NSAID-induced side effects, especially erosions and ulceration of the upper gastrointestinal mucosa.

Pharmaceutically acceptable salts include, for example, salts prepared from pharmaceutically acceptable non-toxic bases including inorganic bases and organic bases. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

Examples of cyclooxygenase-2 selective inhibitors include rofecoxib (VIOXX®, see U.S. Pat. No. 5,474,995, hereby incorporated by reference in its entirety), etoricoxib (ARCOXIA™ see U.S. Pat. No. 5,861,419, hereby incorporated by reference in its entirety), celecoxib (CELEBREX®, see U.S. Pat. No. 5,466,823, hereby incorporated by reference in its entirety), valdecoxib (see U.S. Pat. No. 6,633,272, hereby incorporated by reference in its entirety), parecoxib (see U.S. Pat. No. 5,932,598, hereby incorporated by reference in its entirety), lumiracoxib (PREXIGE®, Novartis), BMS347070 (Bristol Myers Squibb), tiracoxib or JTE522 (Japan Tobacco), ABT963 (Abbott), CS502 (Sankyo) and GW406381 (GlaxoSmithKline).

The term “beta adrenergic receptor blocking agents” and “beta blocker” mean compounds that block beta adrenergic receptors, including pharmaceutically acceptable salts thereof. Such pharmaceutically acceptable salts include those formed from inorganic acids such as hydrochloric, sulfuric and phosphoric acids and those formed from organic acids such as maleic acid, 2-naphthalenesulfonic acid, 3,5-di-tert butylsalicylic acid, 2-chloro-4,6-disulfamoylphenol, 2,5-dihydroxybenzoic acid (gentisic acid), citric acid, pamoic acid, pyruvic acid, isethionic acid, fumaric acid or the like. Beta adrenergic receptor blocking agents are well known in the art and include but are not limited to acebutolol, atenolol, betaxolol, bioprolol, carteolol, labetalol, metoprolol, nadolol, penbutolol, pindolol, propanolol, and timolol. Conventional dosage amounts of beta adrenergic receptor blocking agents can be utilized with the present invention. Such amounts are known in the art and described, for example, in the Physician's Desk Reference. Timolol maleate, for example, may be administered at a dose of about 5 mg, about 10 mg or about 20 mg.

The term “concomitantly administering” means administering the agents substantially concurrently. The term “concomitantly administering” encompasses not only administering the two agents in a single pharmaceutical dosage form but also the administration of each active agent in its own separate pharmaceutical dosage formulation. Where separate dosage formulations are used, the agents can be administered at essentially the same time, i.e., concurrently.

The term “sequentially administering” means administering the agents at separately staggered times. Thus, agents can be sequentially administered such that the beneficial pharmaceutical effect of the beat blocker and COX-2 inhibitor are realized by the patient at substantially the same time. Thus, for example, if a COX-2 selective inhibitor and beta blocker are both administered on a once a day basis, the interval of separation between sequential administration of the two agents can be up to twelve hours apart.

For purposes of this specification, treating migraine means relieving both the headache and the consequent associated symptoms of migraine. Treating migraine is synonymous with the acute treatment of migraines.

For purposes of this specification, prevention of migraine means reducing the severity, the frequency or both the severity and frequency of migraine attacks. Preventing migraines is synonymous with migraine prophylaxis or the chronic treatment of migraines.

For purposes of this specification, migraine is meant to include migraine without aura, migraine with aura, migraine with typical aura, migraine with prolonged aura, familial hemiplegic migraine, basilar migraine, migraine aura without headache, migraine with acute onset aura, ophthalmoplegic migraine, retinal migraine, childhood periodic syndromes that may be precursors to or associated with migraine, benign paroxysmal vertigo of childhood, alternating hemiplegia of childhood, status migrainosus and migrainous infarction. Reference is made to the following: Headache Classification Committee of the International Headache Society: Classification ad diagnostic criteria for headache disorders, cranial neuralgias and facial pain. Cephalalgia. 1988;8(suppl 7):1-96, which is hereby incorporated by reference in its entirety.

For purpose of this specification, an amount that is effective to treat or prevent migraine is that amount that will relieve the subject being treated of the symptoms of the migraine attack and the specific dose level and frequency of dosage may vary and will depend upon a variety of factors including the activity of the specific compounds used in combination, the metabolic stability and length of action of the compounds, the age, body weight, general health, sex diet, mode and time of administration, rate of excretion, the severity of the particular condition and the host undergoing therapy. However, dosage levels of the active ingredients on the order of about 0.01 mg/kg to about 100 mg/kg of body weight per day, typically about 0.1 to about 10 mg/kg, more particularly about 0.2 to about 5 mg/kg and especially about 0.14 to about 3 mg/kg per day are useful in the novel method of treatment. For the treatment or prevention of migraine, the active ingredients may be administered orally, topically, parenterally, by inhalation, spray, rectally or intravaginally in formulations containing pharmaceutically acceptable carriers.

The term parenteral as used herein includes subcutaneous injections, intravenous, intramuscular, intracisternal injection or infusion techniques.

The active agents of the present invention may be in a form suitable for oral use, for example, tablets, troches, lozenges, aqueous or oily suspensions, dispersible powders or granules, emulsions, hard or soft capsules, solutions, syrups and elixirs. Compositions intended for oral use may be prepared according to any method known to the art for the manufacture of pharmaceutical compositions and typically such compositions contain one or more agents selected from the group consisting of sweetening agents, flavoring agents, coloring agents and preservatives in order to provide pharmaceutically elegant and palatable preparations. These excipients may be for example, diluents such as lactose, calcium carbonate, sodium carbonate, calcium phosphate or sodium phosphate; granulating and disintegrating agents, for example, corn starch or alginic acid; binding agents, for example starch, gelatin or acacia, and lubricating agents, for example, magnesium stearate, stearic acid or talc.

The tablets may be uncoated or they may be coated. Coating can be included to delay disintegration and absorption in the gastrointestinal tract and thereby provide a sustained action over a longer period. For example, a time delay material such as glyceryl monostearate or glyceryl distearate may be employed. They may also be coated by the technique described in the U.S. Pat. Nos. 4,256,108; 4,166,452; and 4,265,874 to form osmotic therapeutic tablets for control release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or miscible solvents such as propylene glycol, PEGs and ethanol, or an oil medium, for example peanut oil, liquid paraffin or olive oil.

Aqueous suspensions contain the active material in admixture with excipients suitable for the manufacture of aqueous suspensions. Such excipients are suspending agents, for example sodium carboxymethylcellulose, methylcellulose, hydroxy-propylmethycellulose, sodium alginate, polyvinyl-pyrrolidone, tragacanth and acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl or n-propyl p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxy-ethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain demulcents, preservatives, flavorants and coloring agents.

The pharmaceutical compositions may be in the form of a sterile injectable aqueous or oleaginous suspension. This suspension may be formulated according to the known art using those suitable dispersing or wetting agents and suspending agents which have been mentioned above.

Injectable compositions are typically in the form of sterile solutions or suspensions, which include the active ingredient in a parenterally acceptable diluent. Among these are sterile water, dextrose 5% in water (D5W), Ringer's solution and isotonic saline, as well as mixtures thereof. Cosolvents such as ethanol, propylene glycol or polyethylene glycols may also be used. Sterile, injectable oil is occasionally employed as a solvent or suspending medium in intramuscular preparations. A representative example is peanut oil. In addition, fatty acids such as oleic acid, preservatives, buffers and local anesthetics find use in the preparation of intramuscular injectables.

The combination of active ingredients may also be administered rectally or intravaginally as suppositories. These can be prepared by mixing the drug with a suitable non-irritating excipient which is solid at ordinary room temperature but molten at normal or elevated body temperature. Examples of such materials include cocoa butter and polyethylene glycols.

For topical use, creams, ointments, gels, solutions, suspensions and the like containing the compound are employed. (For purposes of this application, topical application includes mouth washes and gargles, as well as transdermal applications.) Topical formulations are comprised of a pharmaceutical carrier, which may include, e.g., cosolvents, emulsifiers, penetration enhancers, preservatives or emollients.

The combination of the present invention may also be conveniently administered as an orally disintegrating formulation, such as an orally disintegrating tablet, which rapidly dissolves in a patient's mouth without the use of water. The invention can also be administered as a fast dissolve formulation, such as a fast dissolve wafer, that allows for the instantaneous dissolution in the mouth without water. Methods for preparing such formulations are known in the art.

The active agents of the invention may further be administered in combination with other agents for the treatment or prevention of migraine. Such administration may either be in unit dosage form, concomitantly or sequentially. All conventional anti-migraine agents are used in conjunction with the present invention at conventional doses that are determined by the skilled clinician. These compounds are known and normal daily dosages are well established. Typically, the individual daily dosages for these combinations may range from about one-fifth of the minimally recommended clinical dosages to the maximum recommended levels for the entities when they are given alone. Precise dosages are left to the discretion of the physician

Thus, in further aspects, the invention encompasses pharmaceutical compositions for treating or preventing migraines comprising a cyclooxygenase-2 selective inhibitor, a beta adrenergic receptor blocking agent and one or more agents selected from the group consisting of: sumitriptan, naratriptan, zolmitriptan, eleptriptan, almatriptan, rizatriptan, indomethacin, sulindac, etodolac, mefenamic acid, meclofenamic acid, flufenamic acid, tolfenamic acid, etofenamic acid, tolmetin, ketorolac, diclofenac, ibuprofen, naproxen, fenoprofen, ketoprofen, oxaprozin, piroxicam, meloxicam, tenoxicam, lornoxicam, cinnoxicam, sudoxicam, tenoxicam, phenylbutazone, oxyphenbutazone, apazone, azapropazone, nimesulide, diflunisal, nabumetone, aspirin, sodium salicylate, choline, magnesium trisalicylate, salsalate, diflunisal, salicylsalicyclic acid, sulfasalazine olsalazine, ergotamine, ergonovine, ergonovine, mesylates, ergometrine, methylergonovine, methylsergide, metergoline, ergoloid mesylate, dihydroergotamine, dihydroergocomine, dihydroergocristine, dihydroergocryptine, dihydro-α-ergocryptine, dihydro-β-ergocryptine, ergotoxine, ergocornine, ergocristine, ergocryptine, α-ergocryptine, β-ergocryptine, ergosine, ergostine, bromocriptine, amitriptyline, methysergide, propranolol, valproate, verapamil, metoclopramide, prochlorperazine, caffeine, a CGRP antagonist and an NR2B receptor antagonist, in combination with a pharmaceutically acceptable carrier.

Calcitonin gene-related peptide receptor (CGRP) ligands are disclosed, for example, in the following published patent applications: WO 00/18764 published on Apr. 6, 2000, WO 01/10425 published on Feb. 15, 2001, WO 00/55154 published on Sep. 21, 2000, and WO 98/11128 published on Mar. 19, 1998, all of which are hereby incorporated by reference in their entirety.

NR2B receptor antagonists are disclosed, for example, in WO 02/100352, published on Dec. 19, 2002, which is hereby incorporated by reference in its entirety.

In another aspect, the invention encompasses a method for treating or preventing migraines in a mamrnmalian patient in need of such treatment or prevention comprising administering to said patient a a cyclooxygenase-2 selective inhibitor and a beta adrenergic receptor blocking agent, in combination with one or more agents selected from the group consisting of: sumitriptan, naratriptan, zolmitriptan, eleptriptan, almatriptan, rizatriptan, indomethacin, sulindac, etodolac, mefenamic acid, meclofenamic acid, flufenamic acid, tolfenamic acid, etofenamic acid, tolmetin, ketorolac, diclofenac, ibuprofen, naproxen, fenoprofen, ketoprofen, oxaprozin, piroxicam, meloxicam, tenoxicam, lornoxicam, cinnoxicam, sudoxicam, tenoxicam, phenylbutazone, oxyphenbutazone, apazone, azapropazone, nimesulide, diflunisal, nabumetone, aspirin, sodium salicylate, choline, magnesium trisalicylate, salsalate, diflunisal, salicylsalicyclic acid, sulfasalazine olsalazine, ergotamine, ergonovine, ergonovine, mesylates, ergometrine, methylergonovine, methylsergide, metergoline, ergoloid mesylate, dihydroergotamine, dihydroergocornine, dihydroergocristine, dihydroergocryptine, dihydro-α-ergocryptine, dihydro-β-ergocryptine, ergotoxine, ergocomine, ergocristine, ergocryptine, α-ergocryptine, β-ergocryptine, ergosine, ergostine, bromocriptine, amitriptyline, methysergide, propranolol, valproate, verapamil, metoclopramide, prochlorperazine, caffeine, a CGRP antagonist and an NR2B receptor antagonist, in amounts that are effective to treat or prevent migraines. A preferred agent is metoclopramide.

What is claimed is:

1. A method for treating or preventing migraine in a mammalian patient in need thereof comprising concomitantly or sequentially administering to said patient a cyclooxygenase-2 selective inhibitor and a beta adrenergic receptor blocking agent in amounts that are effective for treating or preventing migraine.

2. The method according to claim 1 wherein the cyclooxygenase-2 selective inhibitor is selected from the group consisting of: rofecoxib, etoricoxib, celecoxib, valdecoxib, parecoxib, lumiracoxib, BMS347070, tiracoxib, ABT963, CS502 and GW406381.

3. The method according to claim 2 wherein the cyclooxygenase-2 selective inhibitor is rofecoxib.

4. The method according to claim 3 wherein rofecoxib is administered at a dose of about 12.5 mg or about 25 mg.

5. The method according to claim 2 wherein the cyclooxygenase-2 selective inhibitor is etoricoxib.

6. The method according to claim 5 wherein etoricoxib is administered at a dose of about 60 mg, about 90 mg or about 120 mg.

7. The method according to claim 2 wherein the cyclooxygenase-2 selective inhibitor is celecoxib.

8. The method according to claim 7 wherein celecoxib is administered at a dose of about 100 mg or about 200 mg or about 400 mg.

9. The method according to claim 2 wherein the cyclooxygenase-2 selective inhibitor is valdecoxib.

10. The method according to claim 9 wherein valdecoxib is administered at a dose of about 10 mg or about 20 mg.

11. The method according to claim 1 wherein the beta adrenergic receptor blocking agent is selected from the group consisting of: acebutolol, atenolol, betaxolol, bioprolol, carteolol, labetalol, metoprolol, nadolol, penbutolol, pindolol, propanolol, and timolol, or pharmaceutically acceptable salts thereof.

12. The method according to claim 11 wherein the beta adrenergic receptor blocking agent is timolol maleate.

13. The method according to claim 12 wherein timolol maleate is administered at a dose of about 5 mg, about 10 mg or about 20 mg.

14. A method for treating migraine in a mammalian patient in need thereof comprising concomitantly or sequentially administering to said patient a cyclooxygenase-2 selective inhibitor and a beta adrenergic receptor blocking agent in amounts that are effective for treating migraine.

15. The method according to claim 14 wherein the cyclooxygenase-2 selective inhibitor is rofecoxib and the beta adrenergic receptor blocking agent is timolol maleate.

16. A method for preventing migraine in a mammalian patient in need thereof comprising concomitantly or sequentially administering to said patient a cyclooxygenase-2 selective inhibitor and a beta adrenergic receptor blocking agent in amounts that are effective for preventing migraine.

17. The method according to claim 16 wherein the cyclooxygenase-2 selective inhibitor is rofecoxib and the beta adrenergic receptor blocking agent is timolol maleate.

18. The method according to claim 1 further comprising administering to said patient one or more agents selected from the group consisting of: sumitriptan, naratriptan, zolmitriptan, eleptriptan, almatriptan, rizatriptan, indomethacin, sulindac, etodolac, mefenamic acid, meclofenamic acid, flufenamic acid, tolfenamic acid, etofenamic acid, tolmetin, ketorolac, diclofenac, ibuprofen, naproxen, fenoprofen, ketoprofen, oxaprozin, piroxicam, meloxicam, tenoxicam, lomoxicam, cinnoxicam, sudoxicam, tenoxicam, phenylbutazone, oxyphenbutazone, apazone, azapropazone, nimesulide, diflunisal, nabumetone, aspirin, sodium salicylate, choline, magnesium trisalicylate, salsalate, diflunisal, salicylsalicyclic acid, sulfasalazine olsalazine, ergotamine, ergonovine, ergonovine, mesylates, ergometrine, methylergonovine, methylsergide, metergoline, ergoloid mesylate, dihydroergotamine, dihydroergocornine, dihydroergocristine, dihydroergocryptine, dihydro-α-ergocryptine, dihydro-α-ergocryptine, ergotoxine, ergocornine, ergocristine, ergocryptine, α-ergocryptine, β-ergocryptine, ergosine, ergostine, bromocriptine, amitriptyline, methysergide, propranolol, valproate, verapamil, metoclopramide, prochlorperazine, caffeine, a CGRP antagonist and an NR2B receptor antagonist, in amounts effective to treat or prevent migraines.

19. A pharmaceutical composition comprising a cyclooxygenase-2 selective inhibitor and a beta adrenergic receptor blocking agent in combination with a pharmaceutically acceptable carrier.

20. The pharmaceutical composition according to claim 19 wherein the cyclooxygenase-2 selective inhibitor is rofecoxib and the beta adrenergic receptor blocking agent is timolol maleate.
